(19) United States
(12) Patent Application Publication
(10) Pub. No.: US 2006/0148758 A1
(43) Pub. Date: Jul. 6, 2006

(54) Method for blood glucose control in a mammal by N-acylated glucosamines

(76) Inventor: Tassos P. Anastassides, Kingston (CA)

Correspondence Address: MANELLI DENISON & SELTER, 2000 M STREET NW SUITE 700, WASHINGTON, DC, 20036-3307, US

(21) Appl. No.: 11/324,294

(22) Filed: Jan. 4, 2006

(30) Foreign Application Priority Data
Jan. 6, 2005 (CA) 2,491,763

(51) Int. Cl. A61K 31/7008

(52) U.S. Cl. 514/62

(57) ABSTRACT

A method for a treatment selected from the group consisting of (a) reducing elevated blood glucose, (b) reducing complications of diseases that are associated with elevations of blood glucose, (c) prevention of diseases that develop as a result of elevated blood glucose, (d) controlling glucose metabolism in a mammal by means of an N-acylated hexosamine which is not effectively metabolized to glucose or glucosamine, (e) preventing the development of the metabolic syndrome, and (f) ameliorating the metabolic syndrome in a mammal that has developed the syndrome as a result of an other condition or conditions, such as ovarian failure or the post-menopausal state,
in a mammal, the method comprising administering to the mammal an effective amount of a N-acylated-2-glucosamine derivative of the general formula (I): 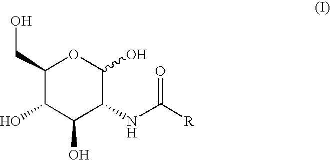
wherein R is an alkyl radical of the general formula CₙH₂ₙ₊₁ wherein n is selected from 2-12; and pharmaceutically-acceptable salts, esters and glucosides thereof.

### FIELD OF INVENTION

The invention relates to (a) reducing elevated blood glucose, (b) reducing complications of diseases that are associated with elevations of blood glucose, (c) prevention of diseases that develop as a result of elevated blood glucose, (d) controlling glucose metabolism by N-acylated glucosamines that are not effectively metabolized to glucose or glucosamine, (e) preventing the development of the metabolic syndrome, and (f) ameliorating the metabolic syndrome once developed, of a mammal, with N-acylated glucosamines, particularly N-butyryl glucosamine.

### BACKGROUND TO THE INVENTION

It is well-known that oral glucose loads result in variable elevations of blood glucose in non-diabetic individuals, and where insulin action was impaired blood glucose rises higher (Reaven G M et al, Diabetes. September 1993;42(9):1324-32). Glucosamine infusion increases plasma glucose levels and simulates some features of diabetes (Monauni T et al, Diabetes. June 2000;49(6):926-35). In consequence of the common use of glucosamine for arthritic complaints, there has been concern that ingestion of relatively high doses of glucosamine may lead to increases in blood glucose, simulating, exacerbating or leading to diabetes. However, ingestion of glucosamine at recommended doses, for example, 1500 mg of glucosamine sulphate per day, over relatively short periods of administration, did not significantly affect blood glucose or serum insulin levels (Tannis A J et al, Osteoarthritis Cartilage. June 2004;12(6):506-11).

There are a number of pathways by which increased blood glucose can damage body tissues. Enzymatic pathways include the conversion of glucose to sorbitol, through the aldose reductase enzyme, and high blood glucose activates different protein kinase forms resulting in complications of blood vessels. Also, there is non-enzymatic glycosylation resulting in advanced glycosylation products which can damage proteins in the body. The hexosamine biosyntesis pathway provides a source of glucosamine-6-phosphate, whose metabolic consequences are thought to be mediated through O-GlcN acetylation. Increased flux through this pathway may promote the complications of diabetes and insulin resistance and some recent studies suggest that a key enzyme in the hexosamine pathway (glutamine:fructose-6-phosphate amidotransferase) may be involved in “cross-talk” between fat cells and muscle (Reviewed in: Buse M G. Am J Physiol Endocrinol Metab January 2006;290(1):E1-8).

It is well known that type II diabetes and insulin resistance is associated with obesity and that obesity is a also risk factor for developing diabetes in children. Increased weight gain and obesity is also commonly seen with ovarian hormonal imbalances, such as occur in women after the menopause, and is frequently linked with metabolic complications such as insulin resistance, lipid abnormalities, hypertension and increased risk of coronary heart disease. This combination of medical problems occurs frequently and is referred to as the “metabolic syndrome”. This syndrome is associated with changes in body mass composition. The number of medical components of the metabolic syndrome in obese post-menopausal women correlates positively with lean body mass, and fat mass, as measured by Dual Energy X-ray Absortiometry (Tongjian You et al. J Clin Endocr Metab 2004 (11) 89, 5517-5522). However, in the quoted work, fat mass did not correlate once adjustment for lean mass and visceral fat was made.

It is known from U.S. Pat. No. 6,479,469B issued Nov. 12, 2002 to Anastassiades, Tassos P. that N-acylated derivatives of glucosamine are useful in the treatment of arthritis; from Canadian Patent Application No. 2,479,632 filed Aug. 31, 2004 by Anastassiades, Tassos P., in the prevention and treatment of osteoporosis and fractures, and International Application No. PCT/CA2004/000034 filed Jan. 14, 2004 to Anastassiades, Tassos P. in ameliorating decreased weight and growth.

### SUMMARY OF THE INVENTION

It is an object of the present invention to provide a method for the reduction of elevated blood glucose.

It is a further object of the invention to provide a method for reducing complications of diseases that are associated with elevations of blood glucose.

It is a further object of the invention to provide a method for the prevention of diseases that develop as a result of elevated blood glucose.

It is a further object to provide a method of controlling glucose metabolism in a mammal by means of an N-acylated hexosamine which is not effectively metabolized to glucose or glucosamine.

It is a further object to provide a method of preventing the development of the metabolic syndrome.

It is a further object to provide a method of ameliorating the metabolic syndrome in an animal that has developed said syndrome as a result of an other condition or conditions, such as ovarian failure or the post-menopausal state.

Accordingly, the invention provides in, one aspect, a method for a treatment selected from the group consisting of

(a) reducing elevated blood glucose,

(b) reducing complications of diseases that are associated with elevations of blood glucose,

(c) prevention of diseases that develop as a result of elevated blood glucose,

(d) controlling glucose metabolism by means of an N-acylated hexosamine which is not effectively metabolized to glucose or glucosamine,

(e) preventing the development of the metabolic syndrome, and

(f) ameliorating the metabolic syndrome in a mammal that has developed said syndrome as a result of an other condition or conditions, such as ovarian failure or the post-menopausal state,
in a mammal, said method comprising administering to said mammal an effective amount of a N-acylated-2-glucosamine derivative of the general formula (I): 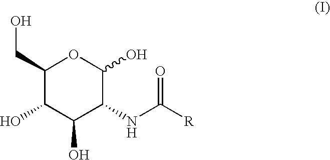
wherein R is an alkyl radical of the general formula CₙH₂ₙ₊₁ wherein n is selected from 2-12; and pharmaceutically-acceptable salts, esters and glucosides thereof.

In preferred embodiments, the invention provides methods as hereinabove, defined wherein said N-acylated 2-glucosamine is N-butyryl-D-glucosamine, of the formula II:

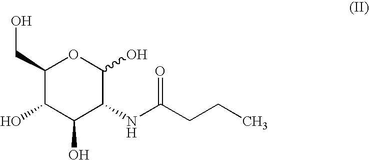

The anomeric and generic structures of formulas (I) and (II), the physical characteristics of the corresponding compounds, the method for their synthesis and tests for purity are described in the aforesaid U.S. Pat. No. 6,479,469B.

The N-acylated derivatives of the general formula (I) may be administered to a mammal in an adequate amount, by one of the following methods, namely, oral, rectal, subcutaneous, trans-dermal, intramuscular, intravenous, or intra-arterial. The derivatives may be mixed with the food or feed to be ingested by the animal, or may be administered in a suitable vehicle, in which the active ingredient is either dissolved or suspended. Solution compositions may be water, salt solutions, other solvents, either alone or in combination with compatible nutrients, antibiotics, drugs suited to the condition, including the medical condition of the mammal.

It will be understood by a person skilled in the art that the active N-acylated glucosamines as hereinbefore defined, should be present and administered in respective, effective and sufficient amounts to alleviate or reverse the high blood glucose, or conditions associated with or as a result of the high blood glucose.

Thus, synthetically prepared N-acetylated glucosamines, as herein defined, administered to mammals, according to the invention, decrease elevated blood glucose of said mammal. The term “mammal” in this specification, particularly includes humans. As a result, the administration of synthetic N-acylated glucosamines is, thus, also useful in diseases that result in complications associated with elevations of blood glucose and the prevention of diseases that result in elevations of blood glucose.

Thus, in a further aspect, the invention provides a method for treating high blood glucose of a mammal, said method comprising of administering to said mammal an effective amount of a compound of the general formula (I), as hereinabove defined.

In a further aspect, the invention provides a method for reducing complications of diseases that are associated with elevations of blood glucose of a mammal, said method comprising of administering to said mammal an effective amount of a compound of the general formula (I), as hereinabove defined.

In a further aspect, the invention provides a method for prevention of diseases that develop as a result of elevated blood glucose of a mammal, said method comprising of administering to said mammal an effective amount of a compound of the general formula (I), as hereinabove defined.

In a further aspect, the invention provides a method for controlling glucose metabolism in a mammal by means of an N-acylated hexosamine which is not effectively metabolized to glucose or glucosamine, said method comprising of administering to said mammal an effective amount of a compound of the general formula (I), as hereinabove defined.

In a further aspect, the invention provides a method for preventing the development of the metabolic syndrome, said method comprising of administering to said mammal an effective amount of a compound of the general formula (I), as hereinabove defined.

In a further aspect, the invention provides a method for ameliorating the metabolic syndrome in an animal that has developed said syndrome as a result of an other condition or conditions, such as ovarian failure or the post-menopausal state, said method comprising of administering to said mammal an effective amount of a compound of the general formula (I), as hereinabove defined.

In a further aspect, the invention provide a method hereinabove defined wherein said effective amount of N-acylated-2-glucosamine is administered to said mammal in a manner selected from the administrative routes consisting of oral, rectal, subcutaneous, trans-dermal, intramuscular, intravenous, or intra-arterial.

In a further aspect, the invention provides a use of a composition comprising an effective amount of N-acylated glucosamine derivative of the general formula (I): 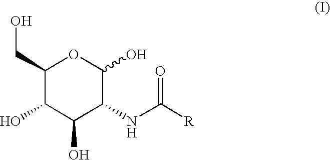
wherein R is an alkyl radical of the general formula CₙH₂ₙ₊₁ wherein n is selected from 2-12; or pharmaceutically-acceptable salts, esters and glucosides thereof; and a physiologically-acceptable diluent or carrier thereof, for a treatment selected from the group consisting of

(a) reducing elevated blood glucose,

(b) reducing complications of diseases that are associated with elevations of blood glucose,

(c) prevention of diseases that develop as a result of elevated blood glucose in a mammal,

(d) controlling glucose metabolism in a mammal by means of an N-acylated hexosamine which is not effectively metabolized to glucose or glucosamine,

(e) preventing the development of the metabolic syndrome, and

(f) ameliorating the metabolic syndrome in a mammal that has developed said syndrome as a result of an other condition or conditions, such as ovarian failure or the post-menopausal state.

In a further aspect, the invention provides a method of manufacturing a medicament intended for a therapeutic application selected from the group consisting of a method of manufacturing a medicament intended for a therapeutic application selected from the group consisting of

(a) reducing elevated blood glucose,

(b) reducing complications of diseases that are associated with elevations of blood glucose,

(c) prevention of diseases that develop as a result of elevated blood glucose,

(d) controlling glucose metabolism in a mammal by means of an N-acylated hexosamine which is not effectively metabolized to glucose or glucosamine,

(e) preventing the development of the metabolic syndrome, and

(f) ameliorating the metabolic syndrome in a mammal that has developed said syndrome as a result of an other condition or conditions, such as ovarian failure or the post-menopausal state,
in a mammal characterized in that said method comprising admixing a N-acetylated-2-glucosamine derivative of the general formula (I): 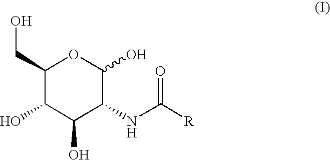
wherein R is an alkyl radical of the general formula CₙH₂ₙ₊₁ wherein n is selected from 2-12; or pharmaceutically-acceptable salts, esters and glucosides thereof; and a physiologically-acceptable diluent or carrier thereof; or pharmaceutically-acceptable compositions thereof; and a physiologically acceptable dilutent or carrier thereof.

In a further aspect, the invention provides use of pharmaceutical composition for the manufacturing of a medicament for a therapeutic application selected from the group consisting of a use of a pharmaceutical composition for the manufacture of a medication for the therapeutic application selected from the group consisting of

(a) reducing elevated blood glucose,

(b) reducing complications of diseases that are associated with elevations of blood glucose,

(c) prevention of diseases that develop as a result of elevated blood glucose,

(d) controlling glucose metabolism in a mammal by means of an N-acylated hexosamine which is not effectively metabolized to glucose or glucosamine,

(e) preventing the development of the metabolic syndrome, and

(f) ameliorating the metabolic syndrome in an animal that has developed said syndrome as a result of an other condition or conditions, such as ovarian failure or the post-menopausal state,
in a mammal characterized in that said method comprising admixing a N-acetylated-2-glucosamine derivative of the general formula (I): 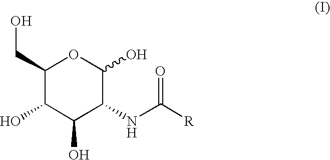
wherein R is an alkyl radical of the general formula CₙH₂ₙ₊₁ wherein n is selected from 2-12; or pharmaceutically-acceptable salts, esters and glucosides thereof; and a physiologically-acceptable diluent or carrier thereof; or pharmaceutically acceptable compositions thereof; and a physiologically acceptable dilutent or carrier thereof.

Preferably, the N-acylated-2-glucosamine in the aforesaid methods and uses according to the invention is N-butyryl-D-glucosamine (II), or pharmaceutically-acceptable salts, esters, glucosides, thereof; or pharmaceutically-acceptable compositions thereof.

### BRIEF DESCRIPTION OF THE DRAWINGS

In order that the invention may be better understood, preferred embodiments will now be described, by way of example only, with reference to the accompanying drawing, wherein

FIG. 1 is a graph of blood glucose concentration levels against time; and

FIG. 2 is a graph of radioactivity levels from oral or IP[3H]GlcNBu incorporated into plasma over time.

### DETAILED DESCRIPTION OF PREFERRED EMBODIMENTS

In the experiments performed and described herein, there were four groups of Spague Dawley type male rats, weighting approximately 300 g each, consisting of four (4) animals in each group. The animals were fed freely on standard rat chow and were then fasted for 24 hours. At the end of this period, said animals were lightly anaesthetized with nitrogen oxide and halothane, the body temperature being maintained with a heat lamp, and the femoral artery was cannulated, with a heparinized catheter and syringe, so that multiple blood samples could be obtained at serial time points. The test compounds were administered by gavaging the animals, said compounds having being dissolved in 3 ml of normal saline. The concentrations of the compounds administered to three groups of animals were: 0.736 M (550 mg in 3 ml of saline) of N-butyryl-D-glucosamine (designated as GlcNBu), or 0.736 M (398 mg in 3 ml of saline) of D-glucose (designated as Glc), or a combination of 0.736 M of GlcNBu and 0.736 M Glc dissolved together in 3 ml of saline. No compound was administered to the fourth group of animals (which consisted of a control group) and animals in this group were gavaged instead with 3 ml of normal saline.

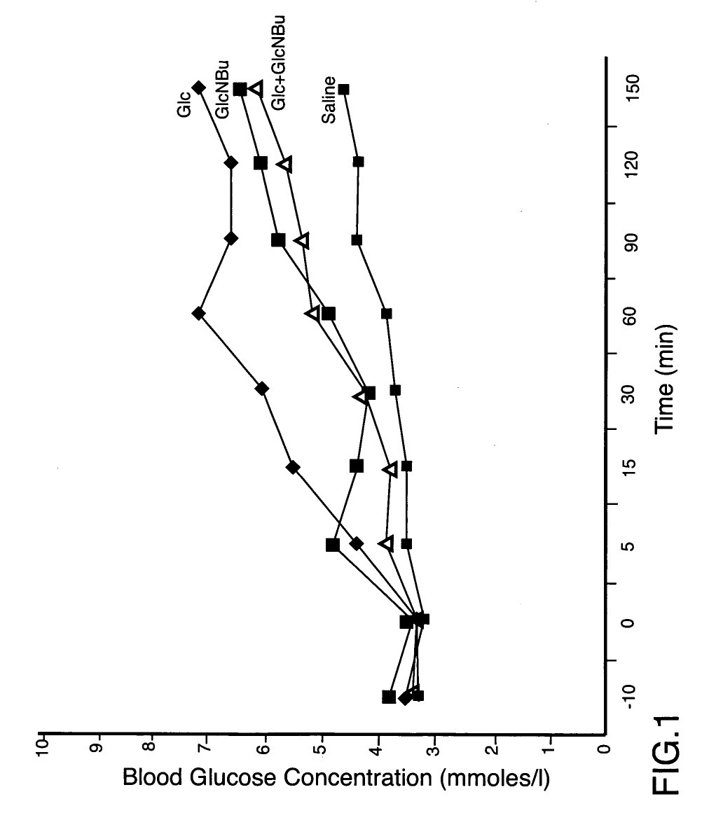

Blood (0.4 ml) was collected at 10 min before the gavage (designated as −10 min in the accompanying FIG. 1), immedietly before the gavage (designated as 0 min) and at the following time points (timed in minutes after time 0) 5, 15, 30, 60, 90, 120 and 150, as shown in the accompanying figure. The glucose concentration in the blood was determined by a commercial glucose oxidase kit, designed for the determination of blood glucose in the blood of patients with diabetes. The determinations were done in triplicate for each time point and for each of the four animals in each of the four groups.

With reference to FIG. 1, each point of the graph represents the mean of the blood glucose concentrations obtained from the four rats in each group of animals. The designations for each group is indicated at the end of the plotted lines and represent, from the top to the bottom of the figure, the mean values measured for the blood glucose for animals that were administered (by gavage) glucose (designated as Glc, small squares), N-butyryl-D-glucosamine (designated as GlcNBu, large squares), glucose and N-butyryl-D-glucosamine administered together (Glc+GlcNBu, triangles) and the saline control (designated as saline, crosses), respectively. It will be observed from the above figure that the blood glucose concentration for all four groups is very similar at −10 minutes before the compounds were administered (approximately between to 3.4 to 3.8 mmoles/l ) and the glucose concentrations remained in said range at 0 minutes, immediately before the compounds were administered. For the animals gavaged with Glc, the blood glucose concentration steadily rose from time 0 to about, 7 mmoles/l, while for the animals gavaged with saline, there was a much smaller increase and slower rise in the blood glucose concentration to about 4 mmoles/l. The increase in blood glucose in rats gavaged with GlcNBu was higher than those gavaged with saline but lower than those rats gavaged with glucose. The pattern of increase in blood glucose of rats gavaged with Glc+GlcNBu together, was similar to those gavaged with GlcNBu alone and the values were generally (after 5 minutes) lower than those of the rats gavaged with Glc alone.

These results indicate that feeding rats with N-butyryl-D-glucosamine results in lower blood glucose, compared to feeding a similar group of rats with an equimolar amount of glucose. Further, feeding the animals with equimolar amounts of glucose and the N-butyryl-D-glucosamine together prevents the expected increase in blood glucose, which would have resulted if the animals were fed with the glucose alone.

These results have been expanded to 6 rats in each group and similar findings to those shown FIG. 1 have been obtained.

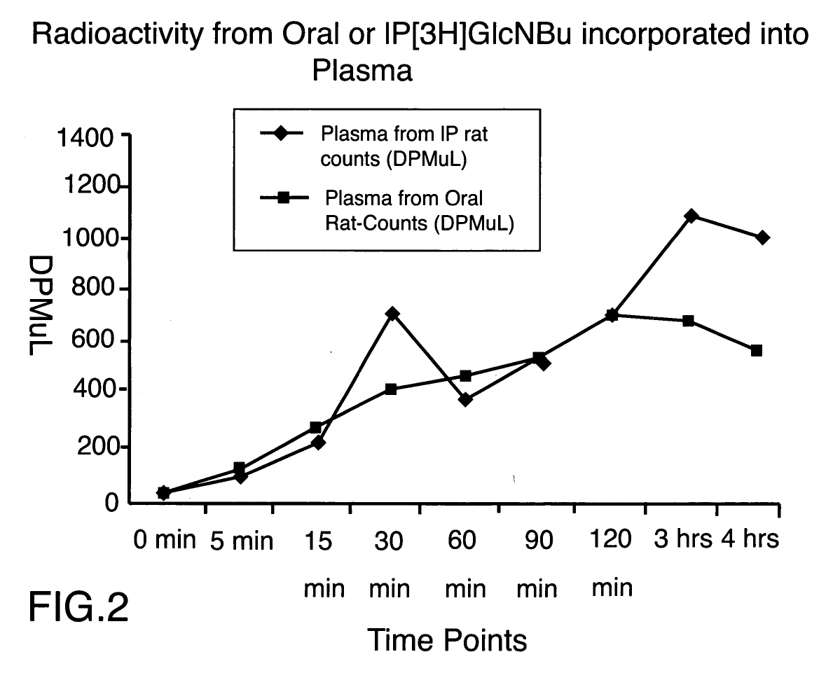

In order to test whether GlcNBu, in fact, was incorporated into serum glucose, or glucosamine, radiolabelled GlcNBu was prepared from [³H] glucosamine and administered either orally or intra-peritoneally (IP) to rats, under identical conditions shown in FIG. 1. Plasma was sampled serially and plasma proteins were precipitated with acetonitrile and the radioactivity into the acetonitrile-soluble fraction containing GlcNBu metabolites was quantified. The results are shown in FIG. 2. It can be seen that the radioactive profile from the oral administration of [³H] GlcNBu is different than the glucose concentration profiles (FIG. 1). Acetonitrile-soluble fractions illustrated in FIG. 2, including the 4 hour time point were analyzed by HPLC, using an amine column and acetonitrile/water mixtures as the mobile phase (1 mL/min flow rate), Absorbance at 195 nm and radioactivity of 2 mL fractions were monitored. Radiolabelled and non-radiolabelled standards of GlcNBu, glucose, glucosamine, N-acetylglucosamine and various disaccharides were used as standards for the column. There was no significant incorporation of radioactivity into glucose, glucosamine or N-acetylglucosamine in the amine column profile. It is, therefore, unlikely that a significant proportion of orally fed GlcNBu is converted to glucose, glucosamine or N-acetylglucosamine, over the time course of the study. Also, glucose or glucosamine are not known to be converted into GlcNBu by animal tissues.

In another experiment, there were four Groups of Sprague Dawley type female rats, with eight animals in each group, of approximately 250 g in weight. Two of the said groups underwent surgical ovariectomy and will be referred as the ovariectomised Groups (OVX). The two remaining groups did not undergo any procedure and will be referred to as the control Groups (C). One C group and one OVX Group were fed GlcNBu, 200 mg/kg/day orally and one C group and one OVX Group were fed an equimolar amount of Glc daily. The daily feedings continued for all of the animals for a period of 6 months. At the end of the 6 month period the animals were euthanized, blood was obtained by cardiac puncture for glucose measurements, 24 hours after the administration of the Glc or GlcNBu, and their body fat and lean body mass was estimated, using a Hologic 4500 Dual Energy X-ray Absortiometry instrument, with small animal software.

It was previously known that ovariectomy increases the body weight and the fat content of animals undergoing this procedure. The results for the lean body mass and the fat content are shown in Table 1 and presented as the mean values±the standard deviations (SD) for each group of Animals.

TABLE 1

| Experimental | Lean body mass | Fat content |
|---|---|---|
| Animal Group | (g, mean ± SD) | (g, mean ± SD) |
| C Glc-fed | 283.93 ± 21.5 | 77.45 ± 21.0 |
| C GlcNBu-fed | 264.02 ± 12.1 | 74.57 ± 23.2 |
| OVX Glc-fed | 296.87 ± 6.2 | 112.94 ± 30.9 |
| OVX GlcNBu-fed | 307.52 ± 34.0 | 126.08 ± 38.4 |

From Table 1 it is apparent that both the lean body mass and the fat content increased in the OVX groups of animals, compared to the C groups. With respect to comparing the C Glc-fed and OVX Glc-fed, there was a 12.9 g increase in lean body mass and a 49.49 g increase in fat content, as a result of the ovariectomies. The proportional (%) mean increase in fat content in the OVX group, compared to lean body mass as a result of the ovariectomies was 4.1% and 39.0%, respectively. There was a 19.91 g decrease in mean lean body mass and a 2.28 g decrease in mean body fat in the C GlcNBu-fed animals compared to the C Glc-fed (non-OVX) animals. This represents a 7.0% decrease in mean lean body mass and a 2.9% decrease in mean body fat as a result of GlcNBu treatment in the non-ovariectomised animals. Thus, treatment with GlcNBu in the non-ovariectomized animals (C), over a six month period, resulted in decreases in both lean body mass and body fat.

For the OVX groups, lean body mass increased by 10.65 g (3.5%) and fat content increased by 13.4 g (11.6%), as a result with GlcNBu treatment.

Although this disclosure has described and illustrated certain preferred embodiments of the invention, it is to be understood that the invention is not restricted to those particular embodiments. Rather, the invention includes all embodiments which are functional or mechanical equivalence of the specific embodiments and features that have been described and illustrated.

I claim:

1. A method for a treatment selected from the group consisting of
(a) reducing elevated blood glucose,
(b) reducing complications of diseases that are associated with elevations of blood glucose,
(c) prevention of diseases that develop as a result of elevated blood glucose,
(d) controlling glucose metabolism by means of an N-acylated hexosamine which is not effectively metabolized to glucose or glucosamine,
(e) preventing the development of the metabolic syndrome, and
(f) ameliorating the metabolic syndrome in a mammal that has developed said syndrome as a result of an other condition or conditions, such as ovarian failure or the post-menopausal state,
in a mammal, said method comprising administering to said mammal an effective amount of a N-acylated-2-glucosamine derivative of the general formula (I): 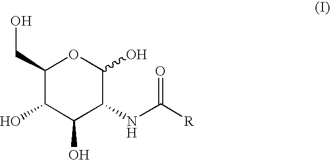
wherein R is an alkyl radical of the general formula CₙH₂ₙ₊₁ wherein n is selected from 2-12; and pharmaceutically-acceptable salts, esters and glucosides thereof.

2. A method as defined in claim 1 of reducing elevated blood glucose of a mammal, comprising of administering to said mammal an effective amount of a N-acylated-2-glucosamine derivative of the general formula (I): 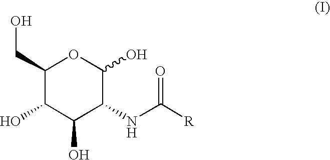
wherein R is an alkyl radical of the general formula CₙH₂ₙ₊₁ wherein n is selected from 2-12; and pharmaceutically-acceptable salts, esters and glucosides thereof.

3. A method as defined in claim 1 for reducing complications of diseases that are associated with elevations of blood glucose of a mammal, comprising of administering to said mammal an effective amount of a N-acylated-2-glucosamine derivative of the general formula (I). 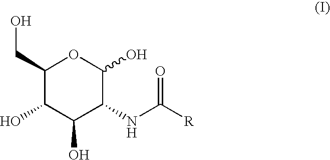
wherein R is an alkyl radical of the general formula CₙH₂ₙ₊₁ wherein n is selected from 2-12; and pharmaceutically-acceptable salts, esters and glucosides thereof.

4. A method as defined in claim 1 for prevention of diseases that develop as a result of elevated blood glucose of a mammal, comprising of administering to said mammal an effective amount of a N-acylated-2-glucosamine derivative of the general formula (I). 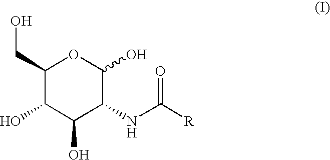
wherein R is an alkyl radical of the general formula CₙH₂ₙ₊₁ wherein n is selected from 2-12; and pharmaceutically-acceptable salts, esters and glucosides thereof.

5. A method as defined in claim 1 for controlling glucose metabolism in a mammal by means of an N-acylated hexosamine which is not effectively metabolized to glucose or glucosamine, comprising of administering to said mammal an effective amount of a N-acylated-2-glucosamine derivative of the general formula (I). 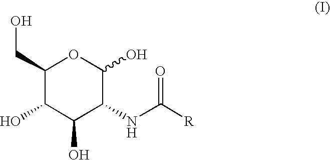
wherein R is an alkyl radical of the general formula CₙH₂ₙ₊₁ wherein n is selected from 2-12; and pharmaceutically acceptable salts, esters and glucosides thereof.

6. A method as defined in claim 1 of preventing the development of the metabolic syndrome, in a susceptible animal, comprising of administering to said mammal an effective amount of a N-acylated-2-glucosamine derivative of the general formula (I): 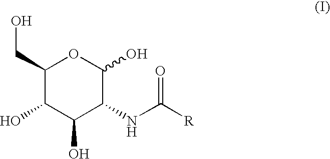
wherein R is an alkyl radical of the general formula CₙH₂ₙ₊₁ wherein n is selected from 2-12; and pharmaceutically acceptable salts, esters and glucosides thereof.

7. A method as defined in claim 1 of ameliorating the metabolic syndrome in an animal that has developed said syndrome as a result of an other condition or conditions, such as ovarian failure or the post-menopausal state, comprising of administering to said mammal an effective amount of a N-acylated-2-glucosamine derivative of the general formula (I): 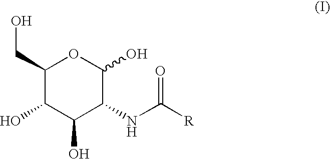
wherein R is an alkyl radical of the general formula CₙH₂ₙ₊₁ wherein n is selected from 2-12; and pharmaceutically acceptable salts, esters and glucosides thereof.

8. A method as defined in claim 1 wherein said N-acylated-2-glucosamine is N-butyryl-D-glucosamine of the formula (II): 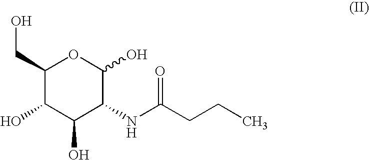
or, pharmaceutically-acceptable salts, esters and glucosides thereof; or pharmaceutically-acceptable compositions thereof.

9. A method as defined in claim 2 wherein said N-acylated-2-glucosamine is N-butyryl-D-glucosamine of the formula (II): 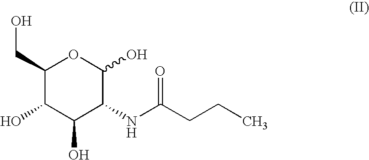
or, pharmaceutically-acceptable salts, esters and glucosides thereof; or pharmaceutically-acceptable compositions thereof.

10. A method as defined in claim 3 wherein said N-acylated-2-glucosamine is N-butyryl-D-glucosamine of the formula (II): 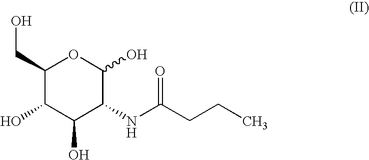
or, pharmaceutically-acceptable salts, esters and glucosides thereof; or pharmaceutically-acceptable compositions thereof.

11. A method as defined in claim 4 wherein said N-acylated-2-glucosamine is N-butyryl-D-glucosamine of the formula (II): 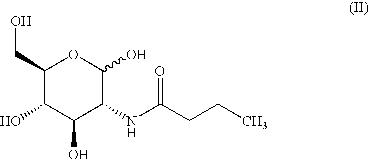
or, pharmaceutically-acceptable salts, esters and glucosides thereof; or pharmaceutically-acceptable compositions thereof.

12. A method as defined in claim 5 wherein said N-acylated-2-glucosamine is N-butyryl-D-glucosamine of the formula (II): 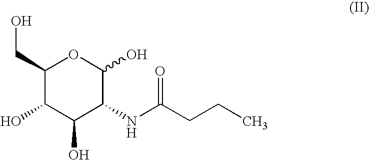
or, pharmaceutically-acceptable salts, esters and glucosides thereof; or pharmaceutically-acceptable compositions thereof.

13. A method as defined in claim 6 wherein said N-acylated-2-glucosamine is N-butyryl-D-glucosamine of the formula (II): 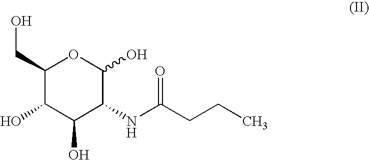
or, pharmaceutically-acceptable salts, esters and glucosides thereof; or pharmaceutically-acceptable compositions thereof.

14. A method as defined in claim 7 wherein said N-acylated-2-glucosamine is N-butyryl-D-glucosamine of the formula (II): 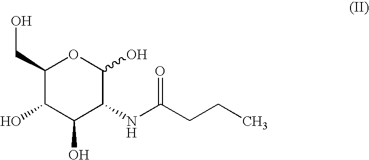
or, pharmaceutically-acceptable salts, esters and glucosides thereof; or pharmaceutically-acceptable compositions thereof.

15. A method as defined in claim 1 wherein said effective amount of said N-acylated-2-glucosamine is administered to said mammal in a manner selected from the administrative routes consisting of oral, rectal, subcutaneous, trans-dermal, intramuscular, intravenous, and intra-arterial.

16. A method as defined in claim 2 wherein said effective amount of said N-acylated-2-glucosamine is administered to said mammal in a manner selected from the administrative routes consisting of oral, rectal, subcutaneous, trans-dermal, intramuscular, intravenous, and intra-arterial.

17. A method as defined in claim 3 wherein said effective amount of said N-acylated-2-glucosamine is administered to said mammal in a manner selected from the administrative routes consisting of oral, rectal, subcutaneous, trans-dermal, intramuscular, intravenous, and intra-arterial.

18. A method as defined in claim 4 wherein said effective amount of said N-acylated-2-glucosamine is administered to said mammal in a manner selected from the administrative routes consisting of oral, rectal, subcutaneous, trans-dermal, intramuscular, intravenous, and intra-arterial.

19. A method as defined in claim 5 wherein said effective amount of said N-acylated-2-glucosamine is administered to said mammal in a manner selected from the administrative routes consisting of oral, rectal, subcutaneous, trans-dermal, intramuscular, intravenous, and intra-arterial.

20. A method as defined in claim 6 wherein said effective amount of said N-acylated-2-glucosamine is administered to said mammal in a manner selected from the administrative routes consisting of oral, rectal, subcutaneous, trans-dermal, intramuscular, intravenous, and intra-arterial.

21. A method as defined in claim 7 wherein said effective amount of said N-acylated-2-glucosamine is administered to said mammal in a manner selected from the administrative routes consisting of oral, rectal, subcutaneous, trans-dermal, intramuscular, intravenous, and intra-arterial.
